(19) United States
(12) Patent Application Publication
(10) Pub. No.: US 2023/0014738 A1
(43) Pub. Date: Jan. 19, 2023

(54) Novel Compositions that Mimic Adipose Tissue in MRI

(71) Applicant: Caliber MRI, Inc., Boulder, CO (US)

(72) Inventors: Elizabeth Mirowski, Nederland, CO (US); Michael Snow, Erie, CO (US)

(73) Assignee: Caliber MRI, Inc., Boulder, CO (US)

(21) Appl. No.: 17/933,109

(22) Filed: Sep. 18, 2022

Related U.S. Application Data
(63) Continuation of application No. 15/481,981, filed on Apr. 7, 2017.
(60) Provisional application No. 62/320,520, filed on Apr. 9, 2016.

(51) Int. Cl. G01R 33/58; G01R 33/48; A61K 47/12

(52) U.S. Cl. CPC G01R 33/58; G01R 33/4828; A61K 47/12; Y10T 436/10; A61K 49/06

(57) ABSTRACT

Provided herein are adipose mimic compositions for use in MRI. The compositions of the invention mimic the MRI properties of human adipose tissue, including T1 relaxation kinetics, T2 relaxation kinetics, magnetic susceptibility, and chemical shift artifact. The compositions of the invention are readily manufactured from inexpensive materials. The compositions of the invention may be used in MRI system calibration or for implementing image correction techniques such as fat suppression.

### CROSS-RELATED APPLICATIONS

This application is a continuation application of U.S. patent application Ser. No. 15/481,981, entitled “Novel Compositions that Mimic Adipose Tissue in MRI,” filed Apr. 7, 2017, which claims the benefit of priority to U.S. Provisional Application Ser. No. 62/320,520, filed Apr. 9, 2016, entitled “Novel Compositions that Mimic Adipose Tissue in MRI,” the contents of which applications are hereby incorporated by reference.

### STATEMENT REGARDING FEDERALLY SPONSORED RESEARCH OR DEVELOPMENT

This invention was made with government support under grant number 70NANB14H297 awarded by the National Institute of Standards and Technology. The government has certain rights in the invention.

### BACKGROUND OF THE INVENTION

Magnetic Resonance Imaging (MRI) is widely used in a number of diagnostic applications. For example, brain, cardiovascular, and orthopedic MRI is commonly used to evaluate various conditions. These evaluations are qualitative in nature, with the gray scale image being interpreted by radiologists to determine the presence and extent of a medical condition.

Accurate identification of adipose tissue is central to many MRI diagnostic methods. In some cases, it is desirable to visualize adipose tissue in an MRI scan, for example in the imaging of lipomas and other conditions comprising fatty elements. In other cases, fat and artifacts caused thereby (e.g. chemical shift artifact) will distort or mask other features desired to be visualized by the scan. In such cases, it is necessary to accurately identify fat and artifacts caused thereby, in order to subtract them from the image.

Various methods for the visualization of adipose tissue and the suppression of adipose tissue artifacts are known. To be effective, these methods depend on the accurate identification of adipose tissue by MRI. Accurate measurement is dependent upon proper calibration of MRI systems and the application of effective algorithms in image construction.

Unfortunately, as with all complex equipment, MRI scanners are imperfect in their measurements. A target may be differently imaged on different MRI machines. Likewise, a single MRI machine may “drift,” over time, with variable readings of the same target at different times. Accordingly, standardization to a known ground truth is essential for accurate MRI performance. In order to calibrate MRI scanners to accurately image adipose, reference standards or mimics are required.

A tissue reference standard, or tissue mimic, is a material that consistently and accurately mimics the MRI-measurable properties of the tissue type it represents. Many MRI measurements are based on T₁ and T₂ relaxation kinetics as well as susceptibility effects, chemical shift effects, and NMR Spectra. For example, the detection of adipose tissue can be accomplished using a T₁-weighted analysis, due to the distinctive and fast T₁ relaxation kinetics of adipose tissue caused by the tightly-bound protons found in lipids.

Adipose mimics for MRI calibration are known in the art. Many of the materials utilized are food products, which share some MRI characteristics with human adipose tissue, including lard (pig fat), safflower oil in a polyurethane mesh, sunflower oil, and grape seed oil. For example, see: PCT Patent Application Serial Number 2012/040611, by Freed et al., entitled “Anthropomorphic, X-Ray, and Dynamic Contrast Enhanced Magnetic Resonance Imaging Phantom for Quantitative Evaluation of Breast Imaging Techniques. In D'Souza et al, 2001, “Tissue mimicking materials for a multi-imaging modality prostate phantom,” Medical Physics 28: 688-700; Bordelois-Boizan et al., “A single phantom to mimic 1H MR spectra of different tissues,” Concepts in Magnetic Resonance Part B: Magnetic Resonance Engineering, Wiley, 2014, 43 (4), pp. 138-145.

These natural products are triglyceride compositions that superficially resemble the MRI characteristics of adipose tissue and which are readily obtained. However, their use is problematic. Food oil and fat products vary considerably from batch to batch in their precise composition as shown in Agiomyrgianaki et al., 2012. “Influence of harvest year, cultivar and geographical origin on Greek EVOO composition: A study by NMR spectroscopy and biometric analysis and Paz et al. 2005 “Characterisation of virgin olive oil of Italian olive cultivars: ‘Frantoio’ and ‘Leccino’, grown in Andalusia.” In Olive Oil, the content of Linoleic acid ranges from 5-10%, Oleic Acid ranges from 70-78% and Palmitic from 10-20%. Other published work shows variations upwards of 30% for various fatty acid constituents.

Such variability in the composition of food products is not unexpected. The concept of terroir is well known to food and wine enthusiasts. Terroir postulates that as a result of differences in soil, climate, and cultivation practices, crops grown in different regions or in different years will having markedly differing character. Terroir effects on food chemical composition have been well documented in the field of food science for crops such as cocoa, grapes, coffee, and other staples. Accordingly, oilseed crops (and animal products) would also be expected to display significant batch-to-batch and season-to-season variability.

Accordingly, the prior art fat mimics based on food products cannot be used as accurate standard materials for MRI calibration due to their lack of consistency in composition. There is therefore a need in the art for novel reference materials with consistent composition that mimic the MRI properties of adipose tissue. Furthermore, because the MRI properties of adipose tissue will vary from organ to organ, there is a need in the art for novel compositions that can mimic diverse forms of adipose.

Provided herein are novel compositions which meet the aforementioned needs, and which provide the art with novel methods of improving the accuracy of MRI imaging.

### SUMMARY OF THE INVENTION

The various embodiments of the invention are directed to methods and compositions of matter useful in the calibration of MRI systems and image construction techniques. The inventions described herein provide the art with novel reference materials of consistent composition that mimic the MRI properties of adipose tissues across several measures, thereby enabling consistent MRI system calibration.

In certain embodiments, the scope of the invention includes compositions comprising liquid blends of fatty acids, which mimic the MRI characteristics of human adipose tissue. In another aspect, the invention encompasses solid forms of adipose mimic compositions.

In another aspect, the scope of the invention encompasses methods of utilizing the adipose mimic compositions provided herein as standard reference materials for the calibration of MRI systems and methods.

In another aspect, the scope of the invention encompasses MRI calibration objects, which are useful in the application of the methods of the invention.

### BRIEF DESCRIPTION OF THE FIGURES

FIG. 1 depicts measured T₁ values of linoleic acid-oleic acid blends, with varying percentages of linoleic acid. Data is presented for both high-field (3 T) as well as low-field (1.5 T) MRI.

FIG. 2 depicts measured T₂ values of linoleic acid-oleic acid blends, with varying percentages of linoleic acid. Data is presented for both high-field (3 T) as well as low-field (1.5 T) MRI.

### DETAILED DESCRIPTION OF THE INVENTION

### Definitions and Setting

Provided herein are novel compositions, which mimic the MRI properties of adipose tissues in the body. In an improvement to the prior art, the novel compositions have consistent and reproducible MRI characteristics and can be manufactured from inexpensive and readily-available materials.

The adipose mimics of the invention comprise simple blends of free fatty acids and do not share the complex makeup of adipose tissues, which are rich in triglycerides, proteins, and other constituents. However, the adipose mimics of the invention were designed by the inventors of the present disclosure to mirror key aspects of the constituent chemistry of adipose tissue, especially with respect to the configuration of C—H bonds. Accordingly, the mimics faithfully recapitulate a broad range of MRI properties of actual adipose tissue. An “MRI property,” as used herein, means a trait or attribute of a material that is measurable by MRI and which is consistent, i.e. is identical across scans when measured using identical scanning conditions.

A first MRI property of the compositions of the invention is its T₁ relaxation value, also referred to herein simply as “T₁ value.” The T₁ value, as known in the art, is the time it takes for a sample's bulk longitudinal magnetization to return to 63% of its original value following perturbation, and is generally thought of as a measure of how tightly protons are bound in a material.

A second MRI property of the compositions of the invention is the T₂ relaxation value, also referred to herein simply as “T₂ value.” The T₂ value, as known in the art, is the time required for the bulk transverse magnetization of a sample to fall to approximately 37% of its initial value, following perturbation.

A third MRI property of the compositions of the invention is the magnetic susceptibility value. A material's magnetic susceptibility, as known in the art, is the degree to which the material will magnetize within an external magnetic field. Magnetic susceptibility of a material can be used in phase contrast imaging, and other techniques, wherein contrast is enhanced by visualizing changes in magnetic susceptibility that are caused by different components of the body.

A fourth MRI property of the compositions of the invention is their NMR spectra. With the exception of the glycerol backbone peak found in the NMR spectrum of adipose tissues, the NMR spectra of the adipose mimic compositions are well matched to those of adipose tissues, allowing the mimics of the invention to be used in NMR methodologies as well.

The compositions of the invention will have properties like those of adipose tissue when measured in a scan. A scan, as used herein, refers to the output of an MRI imaging process, wherein magnetic fields and radio frequencies are applied to a target object, and the resulting radio frequency output is acquired and reconstructed into an image. The scan will comprise various MRI scan parameters selected by a user in performing the scan, including the MRI components, the operations of the various components in performing the scan, and algorithms and techniques used to create images from collected radio frequency data. For example, scan components include factors such as bore temperature, field strength, repetition time, echo time, flip angle, frequency encoding parameters, gradient settings, etc. For example, one scan parameter is the strength of the magnetic field applied to the target. Clinical MRI machines typically apply magnetic field strengths of 1.5 T or 3.0 T. Another scan parameter is the bore temperature. For example, in calibration scans, a common bore temperature is 20° C.

The MRI scan will further encompass various parameters related to generating an image from the radio frequency data collected during the scan. Various algorithms, as known in the art, may be applied to the acquired radio frequency data to convert such data to spatial information, from which an image can be constructed. For example, the applied algorithms may comprise image processing techniques such as fat suppression methods, for example, difference in resonance frequency with water by means of frequency selective pulses (e.g. CHESS); phase contrast techniques; short T₁ relaxation time by means of inversion recovery sequences (e.g. STIR), the Dixon methods, or hybrids of the above, and others known in the art.

MRI scans, as used herein, are understood to be carried out using a suitable MRI system. An MRI system comprises the various common elements of an MRI machine, including magnets, gradient coils, radio frequency generation antennas, radiofrequency receiving antennas, positioning and control systems for orienting and operating the components, data storage devices (e.g. hard drive) for recording measured signals, and one or more processing elements for image generation. The processing elements may comprise any device or combination of networked devices capable of converting acquired radiofrequency data into an image. The processing elements may comprise, for example, embedded systems, general purpose microprocessors, externally located computers, and other computer devices known to the art. Such devices may further comprise non-transitory computer readable storage media, wherein instructions for carrying out image generation functions are encoded.

Adipose Mimics

In a first aspect, the scope of the invention encompasses novel compositions of matter, which may serve as adipose mimics. The various compositions described herein may act as generic adipose mimics, representative of adipose tissue from animal species, for example, humans.

In certain applications, MRI diagnostic procedures are improved by the detection of a specific adipose subtype. Adipose tissues from different parts of the body, from different animal species, or from humans consuming different diets have varying compositions of fatty acids, and as a result, have distinct MRI properties (Rosa T. Branca et al. In vivo NMR detection of diet-induced changes in adipose tissue composition, J. Lipid Res, 2011, 52:833-839. For example, human breast fat has been observed to have a 1.5 T T₁ value of 296 milliseconds, while human abdominal fat was observed to have a T₁ value of about 253 milliseconds (Rakow-Penner et al. Relaxation times of breast tissue at 1.5 T and 3 T measured using IDEAL. J Magn Reson Imaging 2006; 23:87-91; Yong Chen et al., MR Fingerprinting for Rapid Quantitative Abdominal Imaging, 2016, Radiology 279:278-286). To improve the accuracy of MRI diagnostic methods, it would therefore be desirable to utilize mimics having a specific MRI signature representative of the adipose type to be imaged in a particular scan. The compositions of the invention advantageously provide the art with mimics that can be tuned to create precise MRI signatures matched to specific types of adipose tissue.

The adipose mimic compositions of the invention comprise mixtures of fatty acids in various proportions, as follows:

0-70% linoleic acid;

0-100% oleic acid;

0-25% palmitic acid;

0-10% stearic acid; and

0-10% various polyunsaturated fatty acids.

### Percentages for all Compositions Disclosed Herein are Percentages by Weight.

The compositions of the invention preferably utilize fatty acids that are substantially pure, e.g. of 90% or greater purity. Commercially-supplied fatty acids may be much less than 100% pure. For example, impurities such as other polyunsaturated fatty acids (PUFAs) are often present. Typical impurities comprise long-chain fatty acids with more than one double bond present. For example, most of the polyunsaturated fatty acid impurities in 90% oleic acid are of the 18:x type (oleic is 18:1, linoleic is 18:2, linolenic is 18:3) with less than 1% being >C18 and less than 3% being <C18). For the compositions disclosed herein, the purity of linoleic acid is preferably about 99% and the purity of oleic acid is preferably about 90%. The compositions disclosed herein may comprise up to 10% of such polyunsaturated fatty acid impurities without substantial effects on MRI properties.

Liquid Mimic. In one implementation, the invention encompasses liquid adipose mimics. Liquid adipose mimics are conveniently measured and poured into containers, such as MRI calibration objects, as described later herein. The liquid-form adipose tissue mimics comprise a mixture of linoleic and oleic acid. For example, the liquid mimic may comprise:

0-100% Linoleic Acid;

0-100% Oleic Acid;

0-10% various PUFAs; and

no palmitic or stearic acid.

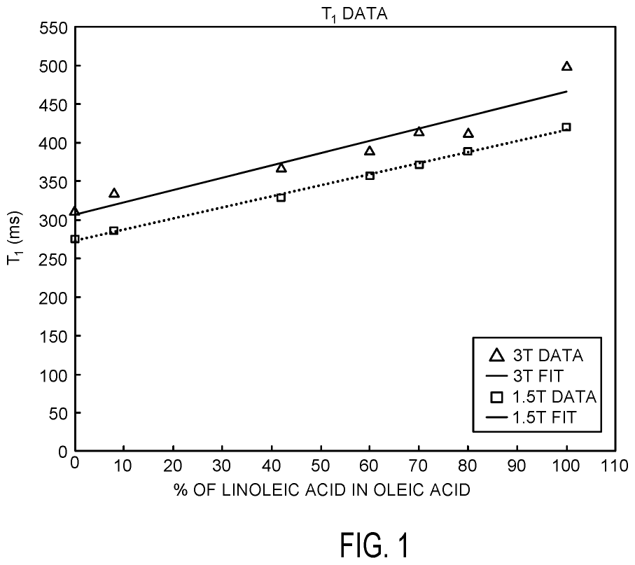

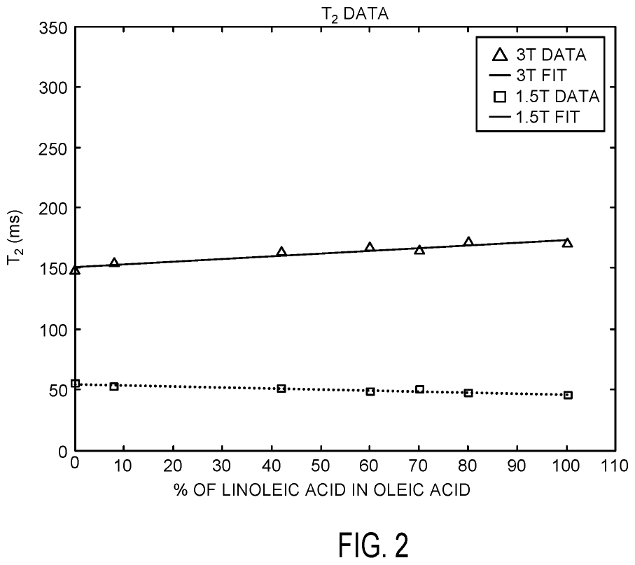

In a blend of oleic and linoleic acid, the T₁ and T₂ values of the blend will vary as a function of the proportion of linoleic acid to oleic acid, as depicted in FIG. 1 and FIG. 2. FIG. 1 shows the relationship between the percentage of linoleic acid in oleic acid and the achieved T₁ at 1.5 T and 3 T. FIG. 2 shows the relationship between the percentage of linoleic acid in oleic acid and the achieved T₂ at 1.5 T and 3 T.

It is well known that T₁ and T₂ vary as a function of temperature. Since the compositions described herein are intended to be used as calibration tools at room temperatures, the T₁ and T₂ values set forth for the disclosed mixtures are those attained at 20° C.±2° C. One of skill in the art can adjust the mixtures disclosed herein to attain desired T₁ and T₂ values at other temperatures.

For example, at 20° C., in a blend of linoleic and oleic acid, the predicted T₁ value when measured at 1.5 T is a function of the percentage of linoleic acid in the blend and can be predicted as follows:

T₁=1.44X+273.1 Equation 1:

wherein T₁ is the resulting T₁ value of the blend when measured at 1.5 T and X is the percentage of linoleic acid in the blend.

In a blend of linoleic and oleic acid, at 20° C., the predicted T₁ value when measured at 3 T is a function of the percentage of linoleic acid in the blend and can be predicted as follows:

T₁=1.59X+307 Equation 2:

wherein T₁ is the resulting T₁ value of the blend when measured at 3 T and X is the percentage of linoleic acid in the blend.

In a blend of linoleic and oleic acid, at 20° C., the predicted T₂ value when measured at 1.5 T is a function of the percentage of linoleic acid in the blend and can be predicted as follows:

T₂=−0.083X+53.9 Equation 3:

wherein T₂ is the resulting T₂ value of the blend when measured at 1.5 T and X is the percentage of linoleic acid in the blend.

In a blend of linoleic and oleic acid, at 20° C., the predicted T₂ value when measured at 3 T is a function of the percentage of linoleic acid in the blend and can be predicted as follows:

T₂=0.22X+151.1 Equation 4:

wherein T₂ is the resulting T₂ value of the blend when measured at 3 T and X is the percentage of linoleic acid in the blend.

Between 0 and 100% linoleic acid, it is possible to achieve specific T₁ and T₂ values, corresponding to those of various adipose tissues throughout the human body, or for adipose tissues from other species. Table 1 lists T₁ and T₂ values for various linoleic acid-oleic acid compositions, at both 1.5 T and 3.0 T.

TABLE 1
T₁ and T₂ values of linoleic-oleic acid blends,
with varying percentages of linoleic acid

| % Linoleic | T₁ at 1.5 T | T₂ at 3T | T₁ at 1.5 T | T₂ at 3 T |
|---|---|---|---|---|
| 0 | 273.1 | 307 | 53.9 | 151.1 |
| 5 | 280.3 | 314.95 | 53.485 | 152.2 |
| 10 | 287.5 | 322.9 | 53.07 | 153.3 |
| 15 | 294.7 | 330.85 | 52.655 | 154.4 |
| 20 | 301.9 | 338.8 | 52.24 | 155.5 |
| 25 | 309.1 | 346.75 | 51.825 | 156.6 |
| 30 | 316.3 | 354.7 | 51.41 | 157.7 |
| 35 | 323.5 | 362.65 | 50.995 | 158.8 |
| 40 | 330.7 | 370.6 | 50.58 | 159.9 |
| 45 | 337.9 | 378.55 | 50.165 | 161 |
| 50 | 345.1 | 386.5 | 49.75 | 162.1 |
| 55 | 352.3 | 394.45 | 49.335 | 163.2 |
| 60 | 359.5 | 402.4 | 48.92 | 164.3 |
| 65 | 366.7 | 410.35 | 48.505 | 165.4 |
| 70 | 373.9 | 418.3 | 48.09 | 166.5 |
| 75 | 381.1 | 426.25 | 47.675 | 167.6 |
| 80 | 388.3 | 434.2 | 47.26 | 168.7 |
| 85 | 395.5 | 442.15 | 46.845 | 169.8 |
| 90 | 402.7 | 450.1 | 46.43 | 170.9 |
| 95 | 409.9 | 458.05 | 46.015 | 172 |
| 100 | 417.1 | 466 | 45.6 | 173.1 |

In one embodiment, the invention comprises a liquid adipose mimic comprising linoleic acid and oleic acid, wherein the percentage of linoleic acid is between 0 and 17.1%

In one embodiment, the invention comprises a liquid composition which mimics human breast adipose tissue, for example having similar T₁ and T₂ values at 1.5 T, comprising a mixture of 9-10% linoleic acid and 80-90% oleic acid, and 0-10% PUFA's. For example, when 90-99% pure linoleic acid and 90% pure oleic acid is used to formulate this mimic, the composition is about 9-10% linoleic acid, 80-81% oleic acid, and about 9-10% PUFA's. For example, in one embodiment, the composition of the invention comprises 10% linoleic acid, 80% oleic acid, and 10% PUFA's.

Solid Mimic. In another aspect, the scope of the invention is directed to solid compositions, which may act as adipose mimics. For example, a solid form adipose tissue mimic may comprise any blend having the following composition:

0-100% linoleic acid;

0-100% oleic acid;

0-50% palmitic acid; and

0-50% stearic acid.

For example, in one embodiment (Solid Composition 1), the invention comprises a solid human adipose tissue mimic comprising:

62% linoleic acid;

25% oleic acid;

10% palmitic acid; and

3% stearic acid

In another embodiment (Solid Composition 2), the invention comprises a solid human adipose mimic comprising:

23% linoleic acid;

49% oleic acid;

22% palmitic acid; and

6% stearic acid

Table 2 shows the T₁ and T₂ values for the solid compositions 1 and 2.

TABLE 2
T₁ and T₂ values for Composition 1 and Composition 2

| | 1.5 T(@20° C.) | | 3.0 T (@20° C.) | |
|---|---|---|---|---|
| | T₁ (ms) | T₂ (ms) | T₁ (ms) | T₂ (ms) |
| Composition 1 | 294 | 44 | 380 | 157 |
| Composition 2 | 286 | 45 | | |

It is noted that palmitic acid and stearic acid by themselves do not produce an MRI signal at either bore temperatures or human body temperatures. In the compositions disclosed above, the major contributors to the T₁ and T₂ values are the linoleic and oleic acids. The palmitic and stearic acid components primarily serve to solidify the mixture at room temperatures.

While the liquid and solid compositions disclosed herein are described with respect to T₁ and T₂ values at 1.5 T and 3.5 T, it will be understood that such compositions will mimic other MRI characteristics of adipose tissue. For example, the mimics of the invention may be utilized in 7 T MRI systems, which are increasingly coming into use.

In one implementation, the mimics of the invention may function as adipose mimics for susceptibility measurements, and will behave as adipose tissue does, with increasing magnetic susceptibility as temperature increases. One of skill in the art can readily assess the magnetic susceptibility of the various mixtures.

Likewise, the mimics of the invention share the adipose NMR spectra of adipose tissues, minus the glycerol backbone peak present in NMR spectra of adipose tissues. One of skill in the art may readily attain NMR spectra for the various fatty acid mixtures disclosed herein.

For example, in one embodiment, one of skill in the art may determine a suitable composition to act as a mimic of a specific type of adipose tissue by determining the T₁ or T₂ value of the specific adipose type at 20° C., and then determining the percentage of linoleic acid in a liquid linoleic-oleic acid blend that has a T₁ or T₂ value matched to that of the specific adipose type of interest, for example as predicted by Equations 1˜4 or Table 1. A composition comprising the selected percentage of linoleic acid may then be manufactured, and the resulting mimic may be used in any number of MRI assays, including T₁-weighted scans, T₂-weighted scans, susceptibility-weighted scans, and NMR analysis (for example, for fat subtraction).

Phase-based magnetic resonance thermometry (MRT) is used for monitoring minimally invasive ablation therapies like focused ultrasound therapy. MRT is prone to errors when applied in tissues with high fat content (e.g. breast tissue, fatty liver) due to heat-induced susceptibility changes. In order to improve accuracy of MRT in the human tissues, reliable knowledge of fat temperature effects on MRI measurements is a prerequisite. Magnetic susceptibility of adipose tissue has been observed to increase with increasing temperature. The adipose mimics of the invention provides a convenient mimic for assessing thermal effects on magnetic susceptibility in adipose tissues. The susceptibility of the adipose mimic compositions of the invention increases linearly in a temperature-dependent manner. Higher proportions of linoleic acid increase the magnitude of the thermal response.

Manufacture and Storage of the Compositions of the Invention. The admixtures of fatty acids comprising the adipose mimics of the invention may be manufactured by simply mixing the components. In the case of compositions comprising linoleic and oleic acid blends, these components are liquid at room temperature, and are readily miscible. They may be combined by stirring, vortexing, or other mechanical means. In the case of the solid compositions, the solid palmitic and stearic acid components must be heated above their melting points (62.9° C. for palmitic acid and 69.3° C. for stearic acid), and then may be thoroughly mixed with the liquid components, after which the admixture is allowed to cool and solidify.

Exposure of the compositions to oxygen and water vapor will cause chemical changes in the compositions of the invention, which will cause shifts in T₁ and T₂ values over time. Accordingly, during manufacture and subsequent storage and use, the compositions should be encased in glass or plastic vessels and any headspace present in the containers should be minimized and comprise an inert gas such as nitrogen or argon rather than air.

Furthermore, heating of the stearic and palmitic acids to melt and blend them with the other constituents causes a chemical reaction that shifts T₁ and T₂ to lower values if oxygen and/or water are present. Accordingly, higher T₁ and T₂ values than set forth for the solid compositions disclosed herein can be attained by heating the solid constituents, and making the admixtures, under vacuum, nitrogen, argon or another inert gas to avoid chemical reactions that can cause an offset in T₁ and T₂ values.

MRI Calibration Objects

The scope of the invention further encompasses MRI calibration objects. An MRI calibration object comprises a container, wherein the container contains an aliquot of an adipose mimic composition. MRI calibration objects are useful in the calibration of MRI systems. The MRI calibration objects can be placed inside an MRI system, allowing measurement of one or more MRI characteristics of the adipose mimic contained within, for purposes of calibrating the system.

A first element of the MRI calibration object is an adipose mimic composition. The adipose mimic composition may comprise any of the liquid or solid adipose mimic compositions disclosed herein.

A second element of the MRI calibration object is a container. The container may comprise any vessel suitable for holding the adipose mimic material and enabling measurement of one or more MRI characteristics of the material. In a preferred implementation, the container comprises a material with a magnetic susceptibility equal or close to that of water. For example, glass, polycarbonate, or other plastics will typically have a susceptibility close to or equal to that of water. In another embodiment, the container comprises a material that is invisible in MRI imaging. For example, materials that lack O—H or C—H bonds, or which have relatively low amounts of such bonds are not readily imaged by MRI. For example, a material such as TEFLON™ is invisible to MRI.

The shape and volume of the container may vary. In various embodiments, the container may be cylindrical, spherical, cuboid, or may comprise another shape. Typical volumes for use in MRI calibration are in the range of 2-1000 ml.

In one embodiment, the size and shape of the container is selected for use in a phantom. A phantom is a shaped container that can be used to calibrate an MRI system. The phantom may comprise a hollow body with a shape matched to the organ or body region of interest, e.g. the head, one or both breasts, the abdomen, the prostate, the pancreas, the liver, etc. The phantom may further comprise an interior frame, made of polycarbonate, which can hold MRI calibration objects comprising mimics of various tissues, e.g. an adipose mimic, a fibroglandular mimic, an interstitial fluid mimic, etc. Scans of the phantom allow imaging of the various mimics for calibration of the system. For example, in one embodiment, the phantom comprises a breast phantom and the adipose mimic composition of the invention is measured in proximity to or admixed with a fibroglandular tissue mimic. In one embodiment, the phantom comprises a breast phantom, wherein fibroglandular mimicking material is admixed with one or more adipose mimicking compositions of the invention, for example, to replicate complex anatomical structures found in human breasts.

The containers of the MRI calibration objects may comprise a void space, i.e., a space not occupied by the adipose mimic composition. Protecting the adipose mimic composition from air and water vapor will prevent drift in its MRI properties. Accordingly, in some embodiments, the void space of the container comprises a vacuum, or an inert gas such as argon or nitrogen.

### Methods of Using Adipose Mimics

The compositions of the invention may be used in various ways. In one aspect, the compositions of the invention are used to calibrate an MRI system. As used herein, “calibration” of an MRI system comprises the basic steps of:

measuring the value of one or more MRI properties of an adipose mimic composition of the invention under selected scan parameters, wherein the value of the one or more MRI properties of the adipose mimic composition, under the selected scan parameters, is known; and comparing the acquired MRI property value of the mimic composition against the expected, known value of such MRI property to determine the degree of variance, if any, between the measured and expected value.

The general method may encompass the measurement of any number of MRI properties of the mimic composition, including T₁, T₂, susceptibility, and NMR spectrum measurements. In one embodiment, the scan comprises a T₁ weighted scan. In one embodiment, the scan comprises a T₂ weighted scan. In one embodiment, the scan comprises a susceptibility-weighted scan. In one embodiment, the scan comprises a phase-based magnetic resonance thermometry scan.

In one embodiment, T₁ values are measured using inversion recovery sequences. In one embodiment, T₂ values are measured using the Carr Purcell Meiboom Gill sequence, performed in an NMR spectrometer of appropriate field strength. An exemplary MRI protocol for determining relaxation values of the adipose mimic of this invention is presented in detail in Keenan et al., 2016 “Design of a breast phantom for quantitative MRI”. Using these standard protocols, the T₁ and T₂ values from newly-developed MR protocols can be directly compared for accuracy and precision.

In some implementations of MRI, it is desirable to perform “fat suppression” or “fat subtraction,” wherein the signal generated by adipose tissue is subtracted from the image to unmask underlying features or to improve resolution. Successful implementation of fat suppression techniques known in the art requires an accurate measurement of fat signals, including signals created directly by adipose tissues and artifacts created by the interface of adipose tissues with other tissues. Fat and water have different resonant frequencies, and as a result, at the interface of fatty tissues and more hydrated tissues, uncorrected MRI images will show ghosting, or a misregistration of fat signals caused by a shift in frequencies. Therefore, in some implementations of MRI, it is desirable to obtain an image at a fat-water boundary in order to quantify chemical shift. Accordingly, in one embodiment of the methods of the invention, the scan is performed wherein an interface exists between water and the adipose mimic of the invention. For example, a sealed vessel containing the adipose mimic of the invention may be placed within a container of water, such that a defined interface exists between the water and the adipose mimic composition. For example, two concentric containers may be used, wherein the inner container and the outer container each hold one component of the fat mimic and water pair. In one embodiment, an interface is created between the adipose mimic of the invention and water, and the scan is a chemical shift imaging scan. In one embodiment, the scan encompasses generation of a fat suppressed image, wherein the degree to which fat has successfully been suppressed from the image is evaluated by viewing an interface between water and the adipose mimic of the invention.

In one embodiment, the scan is a phase-based magnetic resonance thermometry scan, and the measured property of the adipose mimic is magnetic susceptibility. In one embodiment, the measured temperature dependence of an adipose mimic's magnetic susceptibility provides a correction factor for evaluating temperature measurements in a phase-based magnetic resonance thermometry scan.

The methods of the invention may further comprise the additional step of adjusting the MRI system or scan parameters to correct for observed divergence between the measured value and the expected value of the selected MRI property. The adjustment step may comprise any number of affirmative interventions to the MRI scan acquisition process.

In one embodiment, the adjustment comprises a change in the configuration of the MRI system components, such as an alteration of the magnets, coils, antennas, or other physical components. In another embodiment, the adjustment comprises an adjustment to the operating parameters of the MRI system, for example comprising a change in the sequence, frequency, flip angle, timing, or other MRI acquisition parameters. In another embodiment, the adjustment comprises an adjustment to the image construction parameters of the MRI scan. For example, the adjustment may comprise a change in the algorithms used for image construction or error correction, for example, fat suppression techniques. For example, in one embodiment, the adjustment comprises calculation and application of a correction factor to compensate for errors in the imaging process. For example, the adjustment may comprise a renormalization of the historically taken data.

### EXAMPLES

### Example 1. Mimic Composition for Breast Adipose Tissue

The data collected on the adipose tissue mimics of the invention shows T₁, T₂ and susceptibility values that span the values of human adipose tissue cited in literature, namely Rakow-Penner et al. Relaxation times of breast tissue at 1.5 T and 3 T measured using IDEAL. J Magn Reson Imaging 2006; 23:87-91; Yong Chen et al., MR Fingerprinting for Rapid Quantitative Abdominal Imaging, 2016, Radiology 279:278-286; Richard Edden et al. J. Magn REson Imaging. 2010; 32:982-987; S. M Sprinkhuizen et al. Temperature dependence of the magnetic volume susceptibility of human breast fat tissue: an NMR study. Magn Reson Matter Phy. 2012; 25: 33-39 For example, a mimic comprising MRI properties matched to that of breast adipose tissue, for example having matched T₁ and T₂ values, was made. The mimic composition comprises 10% linoleic acid and 90% oleic acid. The mimic material was liquid and was encased in plastic spheres made of a low magnetic susceptibility material. Measurements of T₁ and T₂ were performed on spheres at 1.5 T and 3 T in different MRI machines, using various sequences and scan settings. The mimics showed consistent T₁ and T₂ properties across systems and scan parameters, demonstrating their value as reliable standards.

### Example 2. Susceptibility

Three liquid adipose mimics were made, a 1:9 linoleic-oleic acid blend, a 73:27 linoleic-oleic acid blend, and a 3:1 linoleic-oleic acid blend (ratios are by weight). The magnetic susceptibility shift (Δχ) of the three compositions, relative to water, was measured by an MRI gradient echo sequence at temperatures ranging from 10-50° C. In accordance with human adipose tissue, the magnetic susceptibility of the mimic compositions increased linearly with temperature, with the temperature enhancement effect being greater with increasing linoleic acid content.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1. A method of calibrating an MRI system, comprising the steps of
by the system, measuring the value of a selected MRI property of an adipose mimic composition;
wherein the adipose mimic composition comprises a mixture of:
linoleic acid of at least 90% purity and
oleic acid of at least 90% purity;
wherein the mixture comprises between 5% and 20% linoleic acid;
comparing the acquired value of the selected MRI property of the mimic composition against the expected, known value of such MRI property to determine the degree of variance, if any, between the measured and expected value; and
adjusting the MRI system or scan parameters thereof to correct for any observed divergence between the measured value and the expected value of the selected MRI property.

2. The method of claim 1, wherein
adipose mimic composition comprises 9-10% linoleic acid.

3. The method of claim 1, wherein
the selected MRI property is a T1 relaxation value.

4. The method of claim 1, wherein
the selected MRI property is a T2 relaxation value.

5. The method of claim 1, wherein
the measurement includes a measurement of the selected MRI property of the adipose mimic composition and a measurement of the selected MRI property of the adipose mimic composition wherein an interface exists between water and the adipose mimic composition.

6. The method of claim 1, wherein
the selected MRI property of the adipose mimic composition is magnetic susceptibility.

7. The method of claim 1, wherein,
a temperature dependence of magnetic susceptibility of the adipose mimic composition is measured.

8. The method of claim 1, wherein
the measurement is obtained with the adipose mimic composition in proximity to or admixed with fibroglandular mimicking material.

9. The method of claim 1, wherein
the adipose mimic composition is contained in a container, wherein the container is placed within a phantom.

10. The method of claim 9, wherein
the phantom is a breast phantom.

11. The method of claim 9, wherein
the phantom is selected from the group consisting of: a head phantom, an abdominal phantom, a prostate phantom, a pancreatic phantom, or a liver phantom.
